(19) United States
(12) Patent Application Publication
(10) Pub. No.: US 2022/0226411 A1
(43) Pub. Date: Jul. 21, 2022

(54) PREPARATIONS AND COMPOSITIONS COMPRISING CACTI AND CACTI PARTS FOR ANTIMICROBIAL, ANTIVIRAL AND ANTIFUNGAL ACTIVITY

(71) Applicant: Isaak HAYIK, Yehud (IL)

(72) Inventor: Isaak HAYIK, Yehud (IL)

(21) Appl. No.: 17/577,958

(22) Filed: Jan. 18, 2022

Related U.S. Application Data
(60) Provisional application No. 63/138,536, filed on Jan. 18, 2021.

(51) Int. Cl. A61K 36/33

(52) U.S. Cl. CPC A61K 36/33

(57) ABSTRACT

The invention provides a pharmaceutical composition for treating disorders, diseases and conditions selected from viral diseases, diabetes, lower blood sugar levels in people with type 2 diabetes, high cholesterol, obesity, enlarged prostate and hangovers and viral, bacterial and oxidation related diseases and disorders. The ingredients of the pharmaceutical composition are selected relative amounts of parts and/or extracts of Opuntia ficus-indica that include a selected amount of finely ground fruit or fruit extract; a selected amount of finely ground fruit skin or fruit skin extract; a selected amount of finely ground cladodes; a selected amount of finely ground areoles; a selected amount of finely ground spines and a selected amount of finely ground glochids of Opuntia ficus-indica.

### TECHNICAL FIELD

The present invention pertains to antiviral, antibacterial and antifungal preparations and compositions, which are based on the biome of cacti plants. In particular, the present invention pertains to such preparations and composition that contain extracts of cacti plants that show antimicrobial, antiviral, antiparasitic and antioxidant activity.

### BACKGROUND

Cacti plants are known to be rich in nutrients, which are useful for a healthy human diet. They have also been investigated for phytopathogenic and pathogenic activity, particularly anti-fungal, anti-bacterial reagent in attempts to formulate preparations and compositions for different medical and cosmetic treatments. GAIA SCIENTIA, EDICAO ESPECIAL CACTACEAE, Vol. 9(2): 155-161 describes such activity in a particular cacti species, i.e., Cereus jamacaru DC. subsp. jamacaru, Opuntia ficus-indica, against pathogenic bacteria. Endophyte agents such as Trichoderma longibrachiatum Rifai, Gibberella fujikuroi and Lecythophora decumbens, isolated from C. jamacaru subsp. Jamacaru, demonstrated successful inhibition capabilities of different types of bacteria.

Biotechnological potential of endophytic fungi has been widely observed around the world. This study aimed to evaluate the antibacterial activity of endophytic fungi from cacti Cereus jamacaru DC. subsp. jamacaru, Opuntia ficus-indica (L.) Mill. and Pilosocereus gounellei (F. A. C. Weber) Byles & G. D. Rowley subsp. gounellei of the Brazilian Tropical Dry Forest (Caatinga) against phytopathogenic and pathogenic bacteria to man. The test was performed with 60 endophytes of which 21 (35%) demonstrated activity against at least one pathogenic bacteria to man, especially isolated from C. jamacaru subsp. jamacaru. Trichoderma longibrachiatum Rifai inhibited seven of the 10 bacteria used, followed by Gibberella fujikuroi (Sawada) Wollenw. (6 bacteria) and Lecythophora decumbens (J. F. H. Beyma) E. Weber, Görke & Begerow (5 bacteria) with halos ranging from 5-30 mm. Micrococcus luteus (UFPEDA100) was inhibited by 11 of the 21 tested endophytes. Endophytes did not show activity in the test with phytopathogenic bacteria and with crude extract. Endophytes isolated from cacti growing in Caatinga have potential for producing compounds with antimicrobial activity. ATIVIDADES BIOLOGICAS DE METABOLITOS DE CACTACEAE, E Microccus luteus/Louise Melo De Souza Oliveira, Universidade Federal de Pernambuco, Centro de Biociencias, Programa de Pos Graduacao em Ciencias Biologicas, October 2017, reviews the antibacterial and antioxidant potential of Cereus jamacaru, and to identify the carotenoid pigment of M. Luteus and Cactaceas in Brazil in general. It was observed that this group has antimicrobial, antiviral, antiparasitic and antioxidant activity. It was found that the presence of flavonoids, phenylpropanoids, monoterpenes, triterpenes and steroids was found in methanolic extract of Cereus jamacaru (mandacaru), having bactericidal activity against: Streptococcus mutans, Streptococcus salivarius, Streptococcus sanguis, Streptococcus mitis, Escherichoccus coli and Staph. This extract also showed antioxidant activity.

The present invention pertains to a composition of cacti plants for treating microbes, viruses and fungi with activity that is damaging to human health.

### SUMMARY

In one aspect, the invention provides a pharmaceutical composition for treating disorders, diseases and conditions selected from viral diseases, diabetes, lower blood sugar levels in people with type 2 diabetes, high cholesterol, obesity, enlarged prostate and hangovers.

The following table summarizes the various materials and amounts per 100 gr found in fruits of Opuntia ficus-indica:

TABLE I

| Nutrient | Amount (per 100 gr) | |
|---|---|---|
| Calories | 41 | Kcal |
| Proteins | 0.73 | g |
| Carbohydrates | 9.57 | g |
| Fats | 0.51 | g |
| Saturated Fat | 0.067 | g |
| Cholesterol | 0 | |
| Sodium | 5 | mg |
| Fibers | 3.6 | g |
| Water | 87.55 | g |
| Vitamin A | 2 | μg |
| Vitamin B (total) | 0.6 | mg |
| Vitamin B1 | 0.014 | mg |
| Vitamin B2 | 0.06 | mg |
| Vitamin B3 - Niacin | 0.46 | mg |
| Vitamin B6 | 0.06 | mg |
| Folic acid (Vitamin B9) | 6 | μg |
| Vitamin C | 14 | mg |
| Calcium | 56 | mg |
| Iron | 0.3 | mg |
| Magnesium | 85 | mg |
| Phosphorus | 24 | mg |
| Zinc | 0.12 | mg |
| Potassium | 220 | mg |
| Fatty acids (multi- non-saturated) | 0.213 | g |
| α-Linolenic acid (Omega 3) | 0.023 | g |
| Linoleic acid (Omega 6) | 0.186 | g |
| Fatty acids (non-saturated) | 0.075 | g |
| Oleic acid (Omega 9) | 0.072 | g |

The table above exemplifies the potency of the Opuntia ficus-indica plant and hence their potential as efficient material that has therapeutic effect. Particularly, the invention comprises a pharmaceutical composition that comprises the following parts and/or extracts of Opuntia ficus-indica: a selected amount of finely ground fruit or fruit extract; a selected amount of finely ground fruit skins or fruit skin extract; a selected amount of finely ground cladodes; a selected amount of finely ground areoles; a selected amount of finely ground spines and a selected amount of finely ground glochids of Opuntia ficus-indica, where such composition has antiviral, anti-inflammatory, antioxidant effects in a patient in need of treatment of disorders and diseases related to such effects.

In still another aspect, the pharmaceutical composition of the present invention is suitable for regulating and improving digestion, improving the immune system, optimizing metabolic activity, building strong bones, curing insomnia, and reducing inflammation throughout the body.

In one aspect, the present invention pertains to a pharmaceutical composition that comprises parts and/or extracts of Opuntia ficus-indica comprising: a selected amount of finely ground fruit or fruit extract; a selected amount of finely ground fruit skin or fruit skin extract; a selected amount of finely ground cladodes; a selected amount of finely ground areoles; a selected amount of finely ground spines and a selected amount of finely ground glochids of Opuntia ficus-indica;

an alcoholic, lipid, oily or hydrous vehicle; and

preservatives and additives,

wherein the pharmaceutical composition is suitable for the treatment, prevention, inhibition, prophylaxis and amelioration of diseases and disorders selected from fever, vaginitis, flu, abdominal pain, dryness, arthritis and urinary tract infection, obesity, glycemia and hyperlipidemia, viral diseases, diabetes, lower blood sugar levels in people with type 2 diabetes, high cholesterol, obesity, enlarged prostate and hangovers.

In still another aspect, the present invention pertains to a pharmaceutical composition that comprises the following parts and/or extracts of Opuntia ficus-indica: a selected amount of finely ground fruit or fruit extract; a selected amount of finely ground fruit skin or fruit skin extract; a selected amount of finely ground cladodes; a selected amount of finely ground areoles; a selected amount of finely ground spines and a selected amount of finely ground glochids of Opuntia ficus-indica;

an alcoholic, lipid, oily or hydrous medium; and

preservatives and additives,

wherein said pharmaceutical composition is suitable for the treatment, prevention, inhibition, prophylaxis and amelioration of infections, diseases, conditions and disorders caused, initiated or promoted by microbial, viral or fungal agents.

In still another aspect, the present invention pertains to a pharmaceutical composition that comprises the following parts and/or extracts of Opuntia ficus-indica: a selected amount of finely ground fruit or finely ground fruit extract; a selected amount of finely ground fruit skin or fruit skin extract; a selected amount of finely ground cladodes; a selected amount of finely ground areoles; a selected amount of finely ground spines and a selected amount of finely ground glochids of Opuntia ficus-indica;

an alcoholic, lipid, oily or hydrous medium; and

preservatives and additives,

wherein said composition is suitable for regulating and improving digestion, improving the immune system, optimizing metabolic activity, building strong bones, curing insomnia, and reducing inflammation throughout the body.

Particular viral agents may be Herpes simplex, Equine Herpesvirus, pseudo-rabies virus, flu virus, the syncytial respiratory virus and the human immunodeficiency virus.

Particular bacterial agents may be Streptococcus mutans, Streptococcus salivarius, Streptococcus sanguis, Streptococcus mitis and Escherichia coli e Staphylococcus aureus.

In still another aspect, the present invention pertains to a antimicrobial, antiviral, antiparasitic and antioxidant activity of a pharmaceutical composition that comprises the following parts and/or extracts of Opuntia ficus-indica: a selected amount of finely ground fruit and/or fruit extract; a selected amount of finely ground fruit skin or fruit skin extract; a selected amount of finely ground cladodes; a selected amount of finely ground areoles; a selected amount of finely ground spines and a selected amount of finely ground glochids of Opuntia ficus-indica;

a polymeric matrix;

preservatives and additives,

wherein said composition is suitable for the treatment, prevention, inhibition, prophylaxis and amelioration of diseases, conditions and disorders that express in antimicrobial, antiviral, antiparasitic and antioxidant activity.

In still another aspect, the present invention pertains to a pharmaceutical composition comprising the following parts and/or extracts of Opuntia ficus-indica: a selected amount of finely ground fruit or fruit extract; a selected amount of finely ground fruit skin and/or fruit extract; a selected amount of finely ground cladodes; a selected amount of finely ground areoles; a selected amount of finely ground spines and a selected amount of finely ground glochids of Opuntia ficus-indica, wherein said composition is provided in forms which are suitable for oral administration, such as pills, tablets, capsules, suspensions, syrups, drops, topical administration, intravenous administration and subcutaneous administration.

In one embodiment the composition of the present invention is provided as pharmaceutically acceptable salts, solvates, clathrates, or prodrugs thereof that are useful as for treating, preventing, prophylaxis and amelioration of diseases, conditions and disorders selected from fever, vaginitis, flu, abdominal pain, dryness, arthritis and urinary tract infection, or which are related to antimicrobial, antiviral, antiparasitic and antioxidant activity.

The pharmaceutical compositions of the present invention may be provided for gastrointestinal administration, namely oral in tablets, capsules, drops, syrups or suspensions, rectal as gavage, sublingual, sublabial, administration through the central nervous system, for example, epidural, intracerebral, intracerebroventricular, or administration selected from epicutaneous, intradermal, subcutaneous, nasal, intravenous, intraarterial, intramuscular, intracardiac, intraosseous infusion, intrathecal, intraperitoneal, intravesical, intravitreal, intracavernous injection, intravaginal and intrauterine.

In still another embodiment, the pharmaceutical composition of the present invention is suitable for controlled release in the gastrointestinal tract or in the blood circulation system.

In still another embodiment, the pharmaceutical composition of the present invention is suitable for regular regimen of administration, wherein such regimen may be daily, for example twice a day, weekly, e.g., once every day, or any other regular and repetitive regimen of administration.

In still another embodiment, the active ingredients in the pharmaceutical composition of the present invention are provided in concentration adapted for any particular patient according to different parameters, such as sex, age, weight and medical background.

Table II below details the relative amounts of the active ingredients taken form Opuntia ficus-indica and used in pharmaceutical compositions for treating viral, bacterial and/or oxidation related disorders and diseases and diseases and disorders as detailed above:

TABLE II

| | Range of relative amount of active |
|---|---|
| Ingredients of | ingredients in the pharmaceutical |
| Opuntia ficus-indica | composition (% w/w) |
| Finely ground fruit | 0.5-50 |
| Fruit extract | 0.5-15 |
| Fruit skins | 0.5-25 |
| Fruit skin extract | 0.5-15 |
| Finely ground cladodes | 0.5-25 |
| Finely ground areoles | 0.5-30 |
| Finely ground spines | 0.5-30 |
| Finely ground glochids | 0.5-30 |

Table III below details the relative amounts of ingredients of the pharmaceutical composition that comprises active ingredients taken from Opuntia ficus-indica for treating viral, bacterial and/or oxidation related disorders and diseases and diseases and diseases and disorders as detailed above:

TABLE III

| Ingredient of pharmaceutical | Range of relative amount of |
|---|---|
| composition comp Opuntia | ingredients in the pharmaceutical |
| ficus-indica | composition (% w/w) |
| Active ingredient based on parts and | 5-15 |
| extracts of Opuntia ficus-indica | |
| an alcoholic, lipid, oily or hydrous | 40-75 |
| medium | |
| preservatives and additives | 5-10 |

Table IV below details the relative amounts of the ingredients of a pad that comprises a pharmaceutical composition that comprise active ingredients taken from Opuntia ficus-indica for treating viral, bacterial and/or oxidation related disorders and diseases and diseases and diseases and disorders as detailed above:

TABLE IV

| Ingredient of pharmaceutical | Range of relative amount of |
|---|---|
| composition comp Opuntia | ingredients in the pharmaceutical |
| ficus-indica | composition (% w/w) |
| Active ingredient based on parts and | 5-15 |
| extracts of Opuntia ficus-indica | |
| a polymeric matrix; | 40-75 |
| preservatives and additives | 5-10 |

1. A pharmaceutical composition comprising selected relative amounts of parts and/or extracts of Opuntia ficus-indica comprising: a selected amount of finely ground fruit or fruit extract; a selected amount of finely ground fruit skin or fruit skin extract; a selected amount of finely ground cladodes; a selected amount of finely ground areoles; a selected amount of finely ground spines and a selected amount of finely ground glochids of Opuntia ficus-indica;
an alcoholic, lipid, oily or hydrous vehicle; and
preservatives and additives,
wherein the pharmaceutical composition is suitable for the treatment, prevention, inhibition, prophylaxis and amelioration of diseases and disorders selected from fever, vaginitis, flu, abdominal pain, dryness, arthritis and urinary tract infection, obesity, glycemia and hyperlipidemia, viral diseases, diabetes, lower blood sugar levels in people with type 2 diabetes, high cholesterol, obesity, enlarged prostate and hangovers.

2. The pharmaceutical composition according to claim 1, wherein said composition is provided in forms suitable for oral administration, said forms are selected from pills, tablets, capsules, suspensions, syrups, drops, topical administration, intravenous administration and subcutaneous administration.

3. The pharmaceutical composition according to claim 1, wherein said composition is provided for gastrointestinal administration selected from oral administration in tablets, capsules, drops, syrups or suspensions.

4. The pharmaceutical composition according to claim 5, wherein said composition is provided for controlled release in the gastrointestinal tract or in the blood circulation system.

5. The pharmaceutical composition according to claim 1, wherein said composition is provided in rectal administration.

6. The pharmaceutical composition according to claim 1, wherein said composition is suitable for regular regimen of administration selected from daily, weekly or any other regular and repetitive regimen of administration.

7. The pharmaceutical composition according to claim 1, wherein said selected relative amounts of said Opuntia ficus-indica are 0.5-50% w/w of finely ground fruit and/or 0.5-15% w/w of fruit extract; 0.5-25% w/w of fruit; 0.5-25% w/w of finely ground cladodes; 0.5-30% w/w of finely ground areoles; 0.5-30% w/w of finely ground spines and 0.5-30% w/w of finely ground glochids.

8. The pharmaceutical composition according to claim 1, wherein relative amounts of components of said pharmaceutical composition are 5-15% w/w of active ingredient based on parts and extracts of Opuntia ficus-indica; 40-70% w/w of alcoholic, lipid, oily or hydrous medium; and 5-10% w/w of preservatives and additives.

9. The pharmaceutical composition according to claim 1, wherein said composition is provided in a pad, and wherein relative amounts of said pharmaceutical composition are 5-15% w/w of active ingredient based on parts and extracts of Opuntia ficus-indica; 40-70% w/w of alcoholic, lipid, oily or hydrous medium; and 5-10% w/w of preservatives and additives.

10. A pharmaceutical composition comprising selected relative amounts of parts and/or extracts of Opuntia ficus-indica comprising: a selected amount of finely ground fruit or finely ground fruit extract; a selected amount of finely ground fruit skin or fruit skin extract; a selected amount of finely ground cladodes; a selected amount of finely ground areoles; a selected amount of finely ground spines and a selected amount of finely ground glochids of Opuntia ficus-indica;
an alcoholic, lipid, oily or hydrous medium; and
preservatives and additives,
wherein said composition is suitable for regulating and improving digestion, improving the immune system, optimizing metabolic activity, building strong bones, curing insomnia, and reducing inflammation throughout the body.

11. The pharmaceutical composition according to claim 10, wherein said composition is provided in forms suitable for oral administration, said forms are selected from pills, tablets, capsules, suspensions, syrups, drops, topical administration, intravenous administration and subcutaneous administration.

12. The pharmaceutical composition according to claim 10, wherein said composition is provided for gastrointestinal administration selected from oral administration in tablets, capsules, drops, syrups or suspensions.

13. The pharmaceutical composition according to claim 10, wherein said selected relative amounts of said Opuntia ficus-indica are 0.5-50% w/w of finely ground fruit and/or 0.5-15% w/w of fruit extract; 0.5-25% w/w of fruit; 0.5-25% w/w of finely ground cladodes; 0.5-30% w/w of finely ground areoles; 0.5-30% w/w of finely ground spines and 0.5-30% w/w of finely ground glochids.

14. The pharmaceutical composition according to claim 10, wherein relative amounts of components of said pharmaceutical composition are 5-15% w/w of active ingredient based on parts and extracts of Opuntia ficus-indica; 40-70% w/w of alcoholic, lipid, oily or hydrous medium; and 5-10% w/w of preservatives and additives.

15. The pharmaceutical composition according to claim 10, wherein said composition is provided in a pad, and wherein relative amounts of said pharmaceutical composition are 5-15% w/w of active ingredient based on parts and extracts of Opuntia ficus-indica; 40-70% w/w of alcoholic, lipid, oily or hydrous medium; and 5-10% w/w of preservatives and additives.

16. The pharmaceutical composition comprising selected relative amounts of parts and/or extracts of Opuntia ficus-indica comprising: a selected amount of finely ground fruit or fruit extract; a selected amount of finely ground fruit skin or fruit skin extract; a selected amount of finely ground cladodes; a selected amount of finely ground areoles; a selected amount of finely ground spines and a selected amount of finely ground glochids of Opuntia ficus-indica;
an alcoholic, lipid, oily or hydrous medium; and
preservatives and additives,
wherein said pharmaceutical composition is suitable for the treatment, prevention, inhibition, prophylaxis and amelioration of infections, diseases, conditions and disorders caused, initiated or promoted by microbial, viral or fungal agents.

17. The pharmaceutical composition according to claim 16, wherein said composition is provided in forms suitable for oral administration, said forms are selected from pills, tablets, capsules, suspensions, syrups, drops, topical administration, intravenous administration and subcutaneous administration.

18. The pharmaceutical composition according to claim 16, wherein said selected relative amounts of said Opuntia ficus-indica are 0.5-50% w/w of finely ground fruit and/or 0.5-15% w/w of fruit extract; 0.5-25% w/w of fruit; 0.5-25% w/w of finely ground cladodes; 0.5-30% w/w of finely ground areoles; 0.5-30% w/w of finely ground spines and 0.5-30% w/w of finely ground glochids.

19. The pharmaceutical composition according to claim 16, wherein relative amounts of components of said pharmaceutical composition are 5-15% w/w of active ingredient based on parts and extracts of Opuntia ficus-indica; 40-70% w/w of alcoholic, lipid, oily or hydrous medium; and 5-10% w/w of preservatives and additives.

20. The pharmaceutical composition according to claim 16, wherein said composition is provided in a pad, and wherein relative amounts of said pharmaceutical composition are 5-15% w/w of active ingredient based on parts and extracts of Opuntia ficus-indica; 40-70% w/w of alcoholic, lipid, oily or hydrous medium; and 5-10% w/w of preservatives and additives.
